(19) United States
(12) Patent Application Publication
(10) Pub. No.: US 2025/0256040 A1
(43) Pub. Date: Aug. 14, 2025

(54) A SUB-ASSEMBLY OF A MEDICAMENT DELIVERY DEVICE

(71) Applicant: SHL Medical AG, Zug (CH)

(72) Inventors: Ming-Ting Yin, Taoyuan City (TW); Tai-Fu Wang, Taoyuan City (TW)

(21) Appl. No.: 18/857,657

(22) PCT Filed: Apr. 5, 2023
(86) PCT No.: PCT/EP2023/058989

§ 371 Date: Oct. 17, 2024

(30) Foreign Application Priority Data
Apr. 19, 2022 (EP) 22168804.7

(51) Int. Cl. A61M 5/32; A61M 5/20

(52) U.S. Cl. CPC A61M 5/3202; A61M 5/20; A61M 2005/2006

(57) ABSTRACT

The present disclosure provides a sub-assembly of a medicament delivery device, the sub-assembly comprising: a tubular outer cap extending along a longitudinal axis, an inner cap at least partially surrounded by the tubular outer cap, and a clutch formed by a driving part and a driven part. The inner cap is configured to be removably attached to a housing of the medicament delivery device via a rotational attachment. The driven part is attached to the inner cap and positioned between the tubular outer cap and the inner cap in a direction transverse to the longitudinal axis. The outer cap is axially movable relative to the inner cap between a proximal position and a distal position. The tubular outer cap comprises the driving part extending from a body of the tubular outer cap. The driven part extending in the direction transverse to the longitudinal axis towards the tubular outer cap. The driving part is spaced apart from the driven part in the direction of the longitudinal axis when the tubular outer cap is in the proximal position. The driving part is adjacent to the driven part when the tubular outer cap is in the distal position so that the inner cap can be detached from the housing of the medicament delivery device via a rotation of the outer cap relative to the housing of the medicament delivery device.

### CROSS-REFERENCE TO RELATED APPLICATIONS

The present application is a U.S. National Phase Application pursuant to 35 U.S.C. § 371 of International Application No. PCT/EP2023/058989 filed Apr. 5, 2023, which claims priority to EP patent application Ser. No. 22/168,804.7 filed Apr. 19, 2022. The entire disclosure contents of these applications are herewith incorporated by reference into the present application.

### TECHNICAL FIELD

The present disclosure generally relates to a sub-assembly of a medicament delivery device, and particularly to sub-assembly with a clutch.

### BACKGROUND

Medicament delivery devices such as pen type manual injectors or auto-injectors are generally known for the self-administration of a medicament by patients without formal medical training. For example, patients suffering from diabetes may require repeated injections of insulin, or patients may require regular injections of other types of medicaments such as a growth hormone.

Therefore, a clear operation guide for the patients is important. However, not every user reads a user guide or instructions on a package carefully every time before carrying out a medicament delivery operation, or sometimes unintentional access of the medicament delivery device might happen, e.g., accidentally detaching a cap of the medicament delivery device; or some end users need to perform multiple medicament delivery operations with multiple different medicament delivery devices and will be easily confused or mix up the medicament delivery device operation instructions between different medicament delivery devices.

Therefore, a structural design of the medicament delivery device that can make sure the users follow the operation instruction of the medicament delivery device is desirable.

### SUMMARY

The invention is defined by the appended claims, to which reference should now be made.

In the present disclosure, when the term “distal direction” is used, this refers to the direction pointing away from the dose delivery site during use of the injection device. When the term “distal part/end” is used, this refers to the part/end of the delivery device, or the parts/ends of the members thereof, which during use of the injection device is/are located furthest away from the dose delivery site. Correspondingly, when the term “proximal direction” is used, this refers to the direction pointing towards the dose delivery site during use of the injection device. When the term “proximal part/end” is used, this refers to the part/end of the delivery device, or the parts/ends of the members thereof, which during use of the injection device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a sub-assembly of a medicament delivery device, the sub-assembly comprising: a tubular outer cap extending along a longitudinal axis; and an inner cap at least partially surrounded by the tubular outer cap. The inner cap is configured to be removably attached to a housing of the medicament delivery device via a rotational attachment. A clutch is formed by a driving part and a driven part. The driven part is attached to the inner cap and positioned between the tubular outer cap and the inner cap in a direction transverse to the longitudinal axis. The outer cap is axially movable relative to the inner cap between a proximal position and a distal position. The tubular outer cap comprises the driving part extending from a body of the tubular outer cap. The driven part extends in the direction transverse to the longitudinal axis towards the tubular outer cap. The driving part is spaced apart from the driven part in the direction of the longitudinal axis when the tubular outer cap is in the proximal position. The driving part is adjacent to the driven part when the tubular outer cap is in the distal position so that the inner cap can be detached from the housing of the medicament delivery device via a rotation of the outer cap relative to the housing of the medicament delivery device.

Preferably, according to another embodiment, the driven part extends from a body wall of the inner cap in the direction transverse to the longitudinal axis.

Alternatively, according to another embodiment, the sub-assembly comprises an interlock sleeve. The interlock sleeve comprises a body extending along the longitudinal axis. The interlock sleeve comprises a rotational sleeve fastener engaged with a counter rotational sleeve fastener of the inner cap. The driven part extends from the body of the interlock sleeve in the direction transverse to the longitudinal axis.

Preferably, according to another embodiment, the interlock sleeve comprises an axial sleeve fastener engaged with a counter axial sleeve fastener of the inner cap

Preferably, according to another embodiment, the engagement formed between the rotational sleeve fastener engaged with the counter rotational sleeve fastener of the inner cap is formed by a rib and a counter rib, or is formed by a rib and a groove, or is formed by a non-circular cross-section body and another non-circular cross-section body.

Preferably, according to another embodiment, the engagement formed between the axial sleeve fastener engaged with the counter rotational sleeve fastener of the inner cap is formed by a snap-fit engagement.

Preferably, according to another embodiment, one of the driving and the driven part is a recess, and the other one of the driving part and the driven part is a protrusion that is positioned in the recess when the tubular outer cap is in the distal position.

Preferably, according to another embodiment, the recess faces towards the direction transverse to the longitudinal axis.

Preferably, according to another embodiment, the recess is formed by a ratchet.

Alternatively, according to another embodiment, the clutch engagement formed between the driving part and the driven part is formed by a non-circular cross-section body of the driving part matching with a non-circular cross-section body of the driven part.

Preferably, according to another embodiment, the clutch engagement formed between the driving part and the driven part is a friction-fit engagement.

Preferably, according to another embodiment, the sub-assembly comprising and a retainer.

Preferably, according to another embodiment, the retainer comprises a retainer body and an attachment extending from the retainer body. The attachment is configured to attach to a counter attachment of the medicament delivery device. The inner cap is removable attached to the retainer via the rotational attachment, thereby the inner cap is removable attached to the medicament delivery device via the retainer.

Preferably, according to another embodiment, the attachment of the retainer is one of the snap-fit attachment, a screw thread attachment and a bayonet attachment.

Preferably, according to another embodiment, the rotational attachment is formed by a screw thread of the retainer body and a thread follower of the inner cap that is engaged with the screw thread of the retainer.

Preferably, according to another embodiment, the sub-assembly comprises a delivery member hub configured to attach with a medicament delivery member. The delivery member hub comprises a first engaging member engaged with a first counter engaging member of the retainer, and the delivery member hub comprises a second engaging member engaged with a counter second engaging member of the inner cap. The delivery member hub is axially movable relative to the retainer body between a proximal position and a distal position.

Preferably, according to another embodiment, a rotation of the inner cap relative to the retainer is configured moves the delivery member hub from the proximal position to the distal position.

Preferably, according to another embodiment, an engagement formed between the first engaging member and the counter first engaging member is a screw thread engagement. An engagement formed between the second engaging member and the counter second engaging member is rotational engagement.

Preferably, according to another embodiment, the screw thread engagement between the delivery member hub and the retainer is directed to a reverse longitudinal direction relative to the screw thread engagement between the inner cap and the retainer.

Preferably, according to another embodiment, the delivery member hub is rotationally fixed to the retainer body. One of the second engaging member and the counter second engaging member is a helical cam and the other one of the second engaging member and the counter second engaging member is a cam follower. An engagement formed between the first engaging member and the counter first engaging member is a snap-fit engagement.

Alternatively, according to another embodiment, the outer cap is axially movable relative to the housing of the medicament delivery device from an initial proximal position to the proximal position, and the outer cap is axially immovable relative to the inner cap when the outer cap moves from the proximal position to the proximal position.

Preferably, according to another embodiment, an axial movement of the inner cap relative to the retainer is configured moves the delivery member hub from the proximal position to the distal position.

Preferably, according to another embodiment, the delivery member hub is configured to snap fit to the retainer.

Preferably, according to another embodiment, the sub-assembly comprising a delivery member guard and a lock member. The delivery member guard comprises a proximally directed surface adjacent to a distally directed surface of the tubular outer cap. The delivery member guard comprises a distally directed surface. The lock member comprises a body having a proximally directed surface. The lock member is movable relative to the delivery member guard between a locked position where the proximally directed surface of the lock member is aligned with the distally directed surface of the delivery member guard so that the delivery member guard is prevented from moving distally relative to the lock member and an unlocked position where the proximally directed surface of the lock member is offset to the distally directed surface of the delivery member guard so that the delivery member guard is distally movable relative to the lock member.

Preferably, according to another embodiment, the lock member is rotatable relative to the delivery member guard about the longitudinal axis between the locked position and the unlocked position.

Alternatively, according to another embodiment, the lock member is slidable relative to the delivery member guard in the direction transverse to the longitudinal axis.

Preferably, according to another embodiment, the medicament delivery device is a disposable medicament delivery device.

Alternatively, according to another embodiment, the medicament delivery device is a reusable medicament delivery device.

Preferably, according to another embodiment, the reusable medicament delivery device is formed by a disposable cassette and a reusable drive unit.

Preferably, according to another embodiment, the medicament delivery device is an autoinjector.

Another aspect of the invention provides a cassette unit of a medicament delivery device comprises the sub-assembly.

Preferably, according to another embodiment, the cassette unit comprises the housing of the medicament delivery device. The housing extends along the longitudinal axis. The lock member is axially fixed to the housing.

Preferably, according to another embodiment, the delivery member guard is rotationally fixed to the housing. The housing comprises a bayonet protrusion configured to move along a bayonet track of a drive unit of the medicament delivery device thereby attaching the cassette unit to the drive unit. The lock member comprises a protrusion extending from the body away from the longitudinal axis. The protrusion is next to the bayonet protrusion of the housing in the direction of the longitudinal axis. The protrusion is configured to be rotationally fixed to a wall of the drive unit when the cassette unit is attached to the drive unit.

Preferably, according to another embodiment, the medicament delivery device comprises a multiple-chamber medicament container arranged within the carrier.

Preferably, according to another embodiment, the medicament delivery device is an auto-injector or a manual insulin pen.

Preferably, according to another embodiment, the medicament container is a medicament container without a medicament delivery member.

Preferably, according to another embodiment, the medicament container is a glass cartridge, a plastic cartridge, or a collapsible bag.

Another aspect of the invention provides a method of cap removal of a medicament delivery device, wherein the medicament delivery device comprises the cassette unit and a drive unit, the method comprising the following steps in the following order:
moving the lock member from the locked position to the unlocked position;
moving the tubular outer cap with the delivery member guard in the distal direction relative to the inner cap to the distal position of the tubular outer cap;
rotating the tubular outer cap with the inner cap relative to the housing of the cassette unit, thereby detaching the tubular outer cap and the inner cap from the housing of the cassette unit.

Preferably, according to another embodiment, the step of moving the lock member from the locked position to the unlocked position comprising the step of: rotating the lock member relative to the delivery member guard from the locked position to the unlocked position.

Preferably, according to another embodiment, the step of rotating the lock member relative to the delivery member guard from the locked position to the unlocked position comprises the step of: rotating the lock member relative to the delivery member guard from the locked position to the unlocked position during attaching the cassette unit to a drive unit via a bayonet connection.

Preferably, according to another embodiment, the step of rotating the tubular outer cap with the inner cap relative to the housing comprising the further step of: during rotating the tubular outer cap with the inner cap relative to the housing, moving the delivery member hub from the proximal position to the distal position, thereby mounting a delivery member attached to the delivery member hub to a medicament container with the housing of the medicament delivery device.

The injection devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the injection devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the injection devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-la′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the injection devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIGS. 1-2 schematically show a perspective views of the sub-assembly of the invention.

FIG. 3 schematically shows an inner cap and a delivery member hub of the sub-assembly of FIGS. 1-2.

FIG. 4 schematically shows the inner cap and an interlock sleeve of the sub-assembly of FIGS. 1-2.

FIG. 5 schematically shows the interlock sleeve and a tubular outer cap of the sub-assembly of FIGS. 1-2.

FIGS. 6A-6B schematically show an operation sequence of the sub-assembly of FIGS. 1-2.

FIG. 7 schematically shows a perspective view of the sub-assembly of FIGS. 1-2, a delivery member guard and a lock member.

FIG. 8 schematically shows a perspective view of the delivery member guard and the lock member of FIG. 7.

FIGS. 9A-9B schematically show perspective views of the delivery member guard and the lock member of FIG. 8.

FIG. 10 schematically shows a perspective view of a medicament delivery device with the sub-assembly of the invention.

FIG. 11A-11B schematically shows a show an operation sequence of the medicament delivery device of FIG. 10.

### DETAILED DESCRIPTION

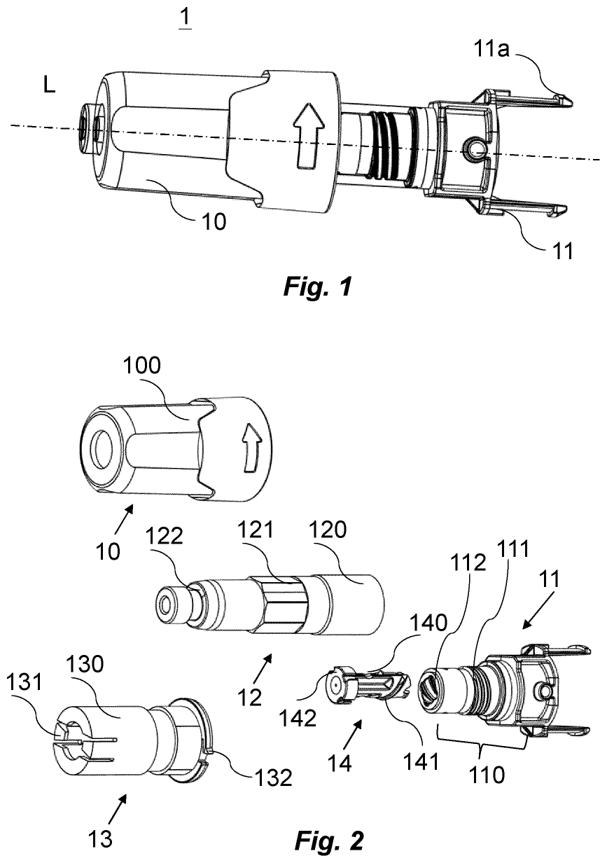

FIGS. 1-6B illustrate a sub-assembly of a medicament delivery device. The sub-assembly 1 comprises a tubular outer cap 10, an inner cap 12, and a clutch.

As shown in FIGS. 1-2, the tubular outer cap 10 extends along a longitudinal axis L between a proximal end and a distal end. The tubular outer cap 10 comprises a body 100. The tubular outer cap 10 is configured to be gripped by a user and is configured to be moved away from the medicament delivery device before a medicament delivery operation. As shown in FIG. 1, the outer cap 10 is generally cylindrical. In one example, an outer surface of the body 100 comprises an indication mark for indicating a user about actions for removing the cap, e.g., rotating. In another example, the outer surface of the body comprises a plurality of recesses and protrusions so that the user can easily grip the outer cap tight. Alternatively, the outer cap can be formed in different shapes dependent on demands, e.g., patient group.

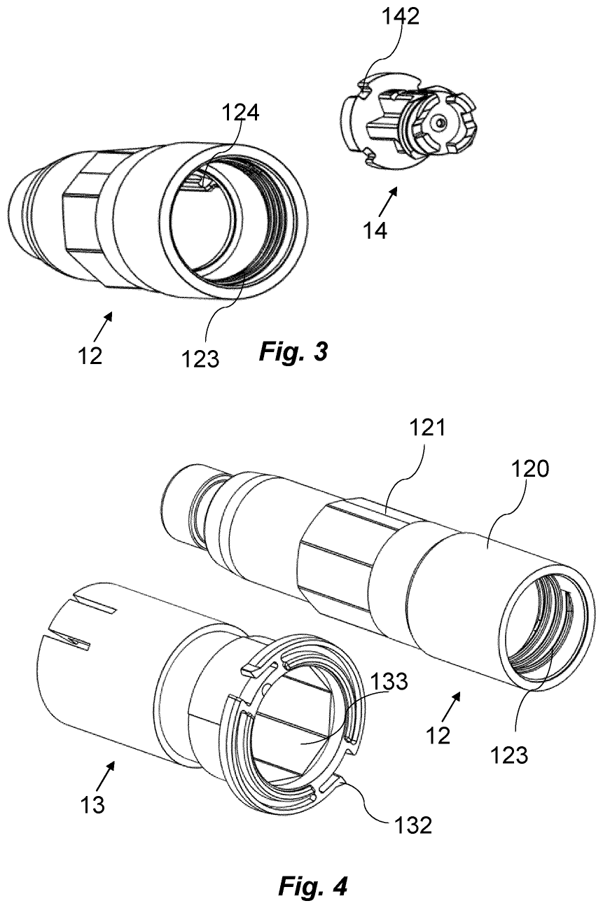

The inner cap 12 comprises a body 120 configured to enclose at least a part of a medicament delivery member of the medicament delivery device. In the example as shown in FIG. 2, the body 120 of the inner cap 12 is generally cylindrical. The body 120 of the inner cap is at least partially surrounded by the tubular outer cap 10. The inner cap 12 is configured to be removably attached to a housing of the medicament delivery device via a rotational attachment. In one example, as shown in FIG. 2, the rotational attachment is a screw thread engagement. In this example, one of the inner cap and the housing of the medicament delivery device comprises a screw thread, and the other one of the inner cap and the housing of the medicament delivery device comprises a thread follower, e.g., a protrusion or a screw thread. In this example, the inner cap 12 comprises a screw thread 123 extending the body 120 of the inner cap, and the housing of the medicament delivery device operably comprises a counter screw thread 111, as shown in FIGS. 2-3. The counter screw thread can be arranged on an integral part of the housing of the medicament delivery device. Alternatively, the counter screw thread can be arranged on another component that is independent to the housing of the medicament delivery device, and is configured to be attached to the housing of the medicament delivery before a use of the medicament delivery device.

In a preferred example, the sub-assembly 1 comprises a retainer 11 comprising a retainer body 110, the counter screw thread 111 extends from an outer surface of the retainer 11 that is configured to be attached to the housing of the medicament delivery device. For example, an attachment 11a extending from the retainer body 110 is configured to attach to a counter attachment of the medicament delivery device, as shown in FIG. 1, the attachment 11a is a snap-fit hook and the counter attachment can be a snap-fit recess/cut-out. Alternatively, the attachment and the counter attachment can be a screw thread and a thread follower, e.g., a protrusion or another matching screw thread.

Alternatively, the rotational attachment can be a bayonet attachment. In this example, one of the inner cap and the housing of the medicament delivery device comprises a bayonet track, and the other one of the inner cap and the housing of the medicament delivery device comprises a bayonet follower, e.g., a protrusion. Similarly, the bayonet track or the bayonet follower can be formed on the retainer 11 that is configured to be attached to the housing, or as an integral part of the housing. The outer cap 10 is axially movable relative to the inner cap 12 between a proximal position and a distal position.

The clutch is formed by a driving part 101 and a driven part 132. The driven part 132 is attached to the inner cap 12 and positioned between the tubular outer cap 10 and the inner cap 12 in a direction transverse to the longitudinal axis L.

The tubular outer cap 10 comprises the driving part 101 extending from a body 100 of the tubular outer cap 10. The driven part 132 extends in the direction transverse to the longitudinal axis towards the tubular outer cap 10. Therefore, when the driving part 101 is adjacent to the driven part 132, the user can rotate the inner cap 12 with the outer cap 10 via the clutch engagement between the driving part 101 and the driven part 132.

The driving part 101 is spaced apart from the driven part 132 in the direction of the longitudinal axis L when the tubular outer cap 10 is in the proximal position so that a rotation of the outer cap 10 around the longitudinal axis L relative to the housing of the medicament delivery device will not transfer to be a rotation of the inner cap 12, in other words, the outer cap 10 will rotate relative to the inner cap 12 when the outer cap is in the proximal position. The driving part 101 is adjacent to the driven part 132 when the tubular outer cap 10 is in the distal position so that the inner cap 12 can be detached from the housing of the medicament delivery device via the rotation of the outer cap 10 relative to the housing of the medicament delivery device.

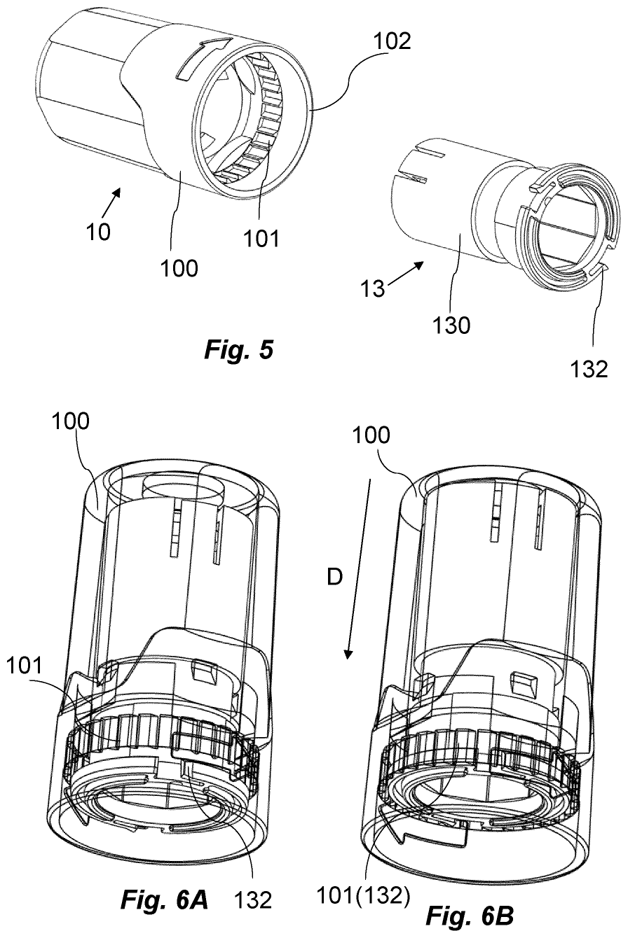

Optionally, the sub-assembly 1 comprises an interlock sleeve 13. The interlock sleeve 13 comprises a body 130 extending along the longitudinal axis L. The interlock sleeve is configured to attach to the inner cap 12 and is positioned between the inner cap 12 and the outer cap 10 in the direction transverse to the longitudinal axis L. In a preferred example, the shape of the body 130 of the interlock sleeve 13 is dependent on the shape of the inner cap 12 and the shape of the outer cap 10. For example, when the outer cap 10 and the inner cap 12 are both shaped to be generally cylindrical, the body 130 of the interlock sleeve 13 is also generally cylindrical, as shown in FIG. 2 and FIG. 5. The interlock sleeve 13 is rotationally immovable relative to the inner cap 12, so that the inner cap 12 can be removed from the housing of the medicament delivery device via a rotation of the interlock sleeve 13 relative to the housing.

Optionally, the interlock sleeve 13 is further axially and immovable relative to the inner cap 12. In one example, the interlock sleeve 13 comprises an axial sleeve fastener 131 engaged with a counter axial sleeve fastener 122 of the inner cap 12, and the interlock sleeve comprises a rotational sleeve fastener 133 engaged with a counter rotational sleeve fastener 121. In one example, the axial sleeve fastener 131 is a snap-fit hook, and the counter axial sleeve fastener 122 is a snap-fit recess, as shown in FIG. 2. In one example, the rotational sleeve fastener 133 of the interlock sleeve is a non-circular shaped cross-section body section of the body 130 of the interlock sleeve 12, and the counter rotational sleeve fastener 121 of the inner cap 12 is a non-circular shaped cross-section body section of the body 120 of the inner cap 12 that matches the rotational sleeve fastener 133 of the interlock sleeve 13, as shown in FIGS. 4-5.

It should be noted that when a non-circular cross-section body of one component is used to match with another non-circular cross-section body of the other component, a rotational engagement is formed, meaning that two components now can only rotate together with one another. For example, the non-circular cross-section body can be a polygonal cross-section body, star shaped cross-section body, or heart shaped cross-section body.

In this example, the driven part 132 extends from the body 130 of the interlock sleeve 13 in the direction transverse to the longitudinal axis L. Alternatively, the driven part extends from the body wall of the inner cap in the direction transverse to the longitudinal axis L, thus, the interlock sleeve doesn't need to be used in this example.

In one example, one of the driving part 101 and the driven part 132 is a recess; and the other one of the driving part 101 and the driven part 132 is a protrusion positioned in the recess when the tubular outer cap 10 is in the distal position. For example, as shown in FIG. 5, the driving part 101 is a recess and the driven part is a protrusion. In one example, the recess faces towards the direction transverse to the longitudinal axis L. In a preferred example, the recess is formed by a ratchet, so that the user will not accidentally tighten the inner cap to the housing of the medicament delivery device by twisting the inner cap in a wrong direction. Alternatively, the clutch engagement formed between the driving part and the driven part is formed by a non-circular cross-section body of the driving part matching with a non-circular cross-section body of the driven part. Alternatively, the clutch engagement formed between the driving part and the driven part is a friction-fit engagement.

The sub-assembly 1 of the invention can be used for a medicament delivery device comprises a medicament container with an integral medicament delivery member. In this example, the sub-assembly 1 prevents the user from accidentally detaching the inner cap, e.g., twisting the inner cap. Alternatively, the sub-assembly 1 can be used for a medicament delivery device comprises a medicament container without a medicament delivery member, e.g., a cartridge or a collapsible bag, especially for the medicament delivery device has a medicament container without a medicament delivery member and a preassembled medicament delivery member, e.g., a needle or a nozzle. The sub-assembly 1 of the invention can be used with a mechanism for establishing fluid communication between the medicament delivery member and the medicament container without a medicament delivery member.

In one example where the sub-assembly comprises the retainer 11, the sub-assembly comprises a delivery member hub 14 configured to attach with a medicament delivery member. The delivery member hub has a body 140 extending along the longitudinal axis L between a proximal end and a distal end. The delivery member is configured to extend through the proximal end of the body 140 of the delivery member hub 14 and the distal end of the body 140 of the delivery member hub 14. In a preferred example, the medicament delivery member is glued to the body 140 of the delivery member hub 14.

In this example, the retainer body 110 comprises a central cavity configured to receive the body of the delivery member hub 14. In this example, the central cavity of the retainer body 110 is configured to align with the medicament container of the medicament delivery device in the direction of the longitudinal axis L, so that the delivery member can be attached to the medicament container through the central cavity. In this example, the delivery member hub 14 comprises a first engaging member 141 engaged with a first counter engaging member 112 of the retainer 11 and a second engaging member 142 engaged with a counter second engaging member 124 of the inner cap 12. The delivery member hub 14 is axially movable relative to the retainer body 110 between a proximal position and a distal position.

In one example, as shown in FIG. 2, a rotation of the inner cap 12 relative to the retainer 11 is configured to move the delivery member hub 14 from the proximal position to the distal position. In one example, an engagement formed between the first engaging member and the counter first engaging member is a screw thread engagement. An engagement formed between the second engaging member and the counter second engaging member is a rotational engagement. In this example, the screw thread engagement between the delivery member hub and the retainer directs to an opposite longitudinal direction to the screw thread engagement between the inner cap and the retainer. For example, the first engaging member 141 is a helical 141 protrusion extending from the body 140 of the delivery member hub 14, and the counter first engaging member 112 is a screw thread 112 extending from an inner surface of the central cavity of the retainer body 110, as shown in FIG. 2. The second engaging member 142 is a cut-out in a wall of the body 140 of the delivery member hub 14, preferably at the proximal end of the body 140 of the delivery member hub 14, and the counter second engaging member 124 is a protrusion 124 extending from an inner surface of body 120 of the inner cap 12, as shown in FIG. 3. Therefore, when the inner cap 12 is rotated relative to the retainer 11 to be detached from the retainer 11, the delivery member hub 14 is rotated, in an opposite direction, with the delivery member. Thereby, the delivery member hub 14 and the delivery member are moved in the distal direction of the outer cap to attach with the retainer 11 and establish the fluid communication with the medicament container of the medicament delivery device.

Alternatively, the delivery member hub is rotationally fixed to the retainer body. In this example, one of the second engaging member and the counter second engaging member is a helical cam and the other one of the second engaging member and the counter second engaging member is a cam follower. Furthermore, an engagement formed between the first engaging member and the counter first engaging member is a snap-fit engagement. Therefore, the rotation of the inner cap relative to the retainer moves the delivery member hub from the proximal position to the distal position and thereby establishing the fluid communication with the medicament container of the medicament delivery device and the delivery member.

Alternatively, the rotation of the inner cap can be only used to detach the inner cap from the retainer. In this example, the inner cap comprises a distally directed surface adjacent to a proximally directed surface of the delivery member hub, and the outer cap is axially immovable relative to the inner cap between an initial proximal position and the proximal position. For example, the outer cap comprises a bump extending in the direction transverse to the longitudinal axis from the inner surface of the outer cap and the inner cap comprises a counter bump engaging with the bump of the outer cap when the outer cap is in the initial proximal position and disengages with the bump of the outer cap when the outer cap is in the proximal position. In a preferred example, the interface between the bump and the counter bump is beveled relative to the longitudinal axis L. In this example, one of the first engaging member of the hub and the counter first engaging member of the retainer is a snap-fit hook and the other one of the first engaging member of the hub and the counter first engaging member of the retainer is a recess/cut-out. In this example, when the user pushes the outer cap in the distal direction relative to the housing of the medicament delivery device, the outer cap, the inner cap and the delivery member hub move all together in the distal direction relative to the retainer until the delivery member hub snap-fits the retainer. When the delivery member hub snap-fits the retainer, the delivery member hub cannot move in the distal direction relative to the retainer and thus, the inner cap cannot move in the distal direction relative to the retainer. Therefore, if the user keeps pushing the outer cap in the distal direction relative to the housing of the medicament delivery device, the bump of the outer cap moves past the counter bump of the inner cap. Preferably, in this example, the engagement between the inner cap and the retainer is a track and protrusion engagement. For example, the inner cap comprises a protrusion positioned within a track of the retainer. The track comprises a longitudinal section extending along the longitudinal axis between a proximal end and a distal end and a circumferential section extending around the longitudinal axis between a first end and a second end. The proximal end of the longitudinal section of the track is a closed end and the distal end of the longitudinal section of the track connects to the first end of the circumferential section. The second end of the circumferential section is an open end where the protrusion can move out from the track at the second end.

In this example, when the outer cap moves from the initial proximal position to the proximal position, the bayonet protrusion of the inner cap moves along a longitudinal section of the bayonet track of the retainer. The inner cap is immovable relative to the retainer when the outer cap moves further from the proximal position to the distal position, and when the outer cap is in the distal position, the outer cap rotationally engages with the inner cap via the clutch as mentioned above. Therefore, the user can rotate the outer cap, thereby the protrusion of the inner cap moves along the circumferential section of the track from the first end of the second end so that the inner cap is detached from the retainer.

It should be noted that the structures on the retainer can be arranged on the housing of the medicament delivery device dependent on the design. When the sub-assembly 1 is used with this type of the medicament delivery device, the sub-assembly does not have the retainer.

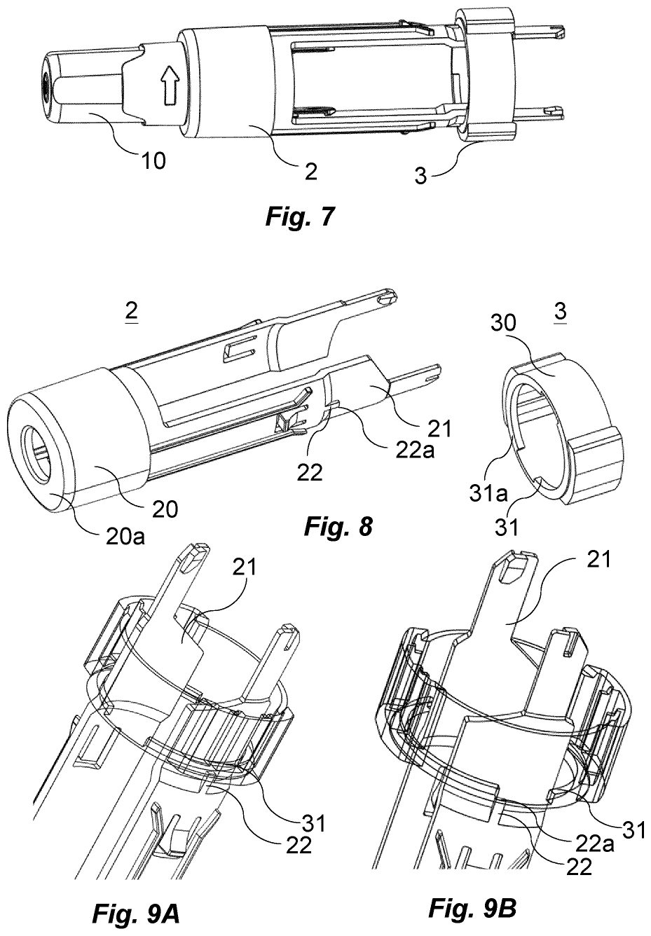

Furthermore, the sub-assembly optionally comprises a secondary safety mechanism. In this example, the sub-assembly comprises a delivery member guard 2 and a lock member 3, as shown in FIG. 7. In a preferred example, the delivery member guard comprises a tubular proximal section 20, and a distal body section 21. The distal body section can be a distally extended arm extending from the tubular proximal section 20 as shown in FIG. 8. Alternatively, the distal body section can be cylindrical. The delivery member guard 2 comprises a proximally directed surface 20a adjacent to a distally directed surface 102 of the tubular outer cap 10. In a preferred example, the proximally directed surface 20a is defined by a proximal end of the tubular proximal section of the delivery member guard 2. Alternatively, the delivery member guard comprises a protrusion extending from an outer surface of the tubular proximal section in the direction transverse to the longitudinal axis, the proximally directed surface is defined by the protrusion. The delivery member guard 2 comprises a distally directed surface 22a. The lock member 3 comprises a body 30 having a proximally directed surface 31a. The lock member 3 is movable relative to the delivery member guard 2 between a locked position where the proximally directed surface 31a of the lock member 3 is aligned with the distally directed surface 22a of the delivery member guard 2 so that the delivery member guard 2 is prevented from moving distally relative to the lock member 3, as shown in FIG. 9A, and an unlocked position where the proximally directed surface 31a of the lock member 3 is offset to the distally directed surface 22a of the delivery member guard 2, as shown in FIG. 9B, so that the delivery member guard 2 is distally movable relative to the lock member 3. Therefore, when the lock member 3 is in the locked position, the outer cap 10 cannot be moved from the proximal position to the distal position. In one example where the outer cap comprises the initial proximal position, the outer cap also cannot be moved from the initial proximal position to the proximal position. In one example, the delivery member guard 2 comprises a rib 22 extending from the distal body section of the delivery member guard 2 in the direction transverse to the longitudinal axis L.

In one example, the lock member 3 is rotatable relative to the delivery member guard 2 about the longitudinal axis L between the locked position and the unlocked position. For example, the lock member 3 can be a rotatable knob. In another example, the lock member is linearly slidable in the direction transverse to the longitudinal axis L from the locked position and the unlocked position. For example, the lock member can be a removable pin configured to be removed by the user before use.

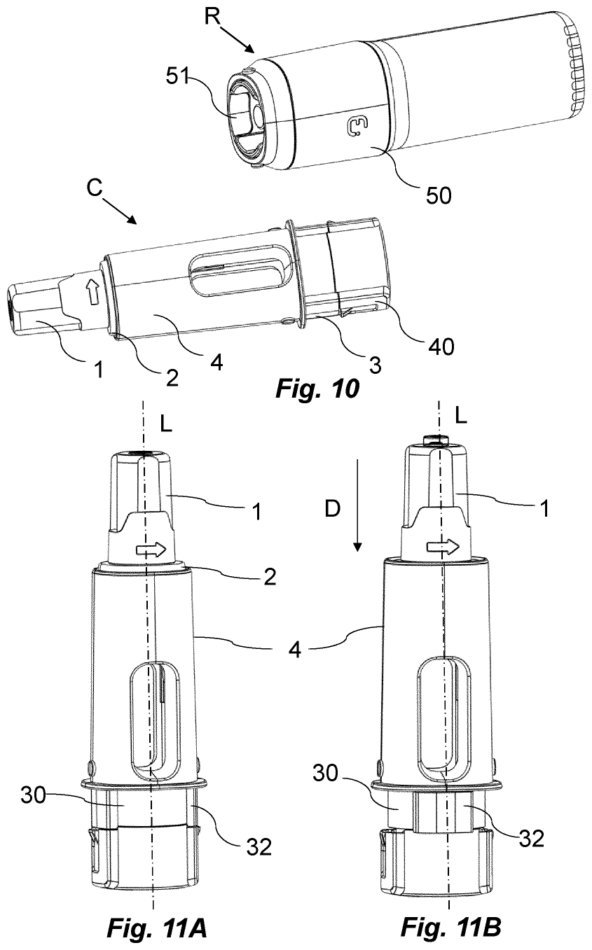

The sub-assembly can be used in a disposable medicament delivery device, e.g., a handhold autoinjector or an on-body injection pump. Alternatively, the sub-assembly can be used in a reusable medicament delivery device that is formed by a disposable cassette unit C and a reusable powerpack R, as shown in FIG. 10. The disposable cassette unit C is configured to accommodate the medicament container. In one example where the sub-assembly is used in the reusable medicament delivery device, the sub-assembly is used in the disposable cassette unit C. For example, the cassette unit comprises the housing 4 of the medicament delivery device. The housing 4 extends along the longitudinal axis L. In this example, the lock member 3 is axially fixed to the housing 4. In a preferred example, the delivery member guard 2 is rotationally fixed to the housing 4. In a preferred example, the housing 4 comprises a bayonet protrusion 40 configured to move along a bayonet track of a drive unit R of the medicament delivery device thereby attaching the cassette unit C to the drive unit R. The lock member 3 comprises a protrusion 32 extending from the body 30 away from the longitudinal axis L. The protrusion 32 is next to the bayonet protrusion 40 of the housing 4 in the direction of the longitudinal axis L. The protrusion 32 is configured to be rotationally fixed to a wall of the drive unit R when the cassette unit is attached to the drive unit R.

The sub-assembly can be operated with the reusable medicament delivery device with the following steps: moving the lock member 3 from the locked position to the unlocked position; moving the tubular outer cap 10 with the delivery member guard 2 in the distal direction relative to the inner cap 12 to the distal position of the tubular outer cap 10; and rotating the tubular outer cap 10 with the inner cap 12 relative to the housing 4 of the cassette unit, thereby detaching the tubular outer cap 10 and the inner cap 12 from the housing 4 of the cassette unit. Furthermore, the step of moving the lock member 3 from the locked position to the unlocked position comprises the step of: rotating the lock member 3 relative to the delivery member guard from the locked position to the unlocked position. In one example where the cassette unit C connects to the drive unit R via a bayonet connection, the step of rotating the lock member 3 relative to the delivery member guard 2 from the locked position to the unlocked position comprising the step of: rotating the lock member 3 relative to the delivery member guard 2 from the locked position to the unlocked position during attachment of the cassette unit C to a drive unit R via a bayonet connection. Furthermore, when the sub-assembly is used with the mechanism for establishing fluid communication between the medicament delivery member and the medicament container without a medicament delivery member, the step of rotating the tubular outer cap 10 with the inner cap 12 relative to the housing 4 comprising the further step of: during rotating the tubular outer cap 10 with the inner cap 12 relative to the housing 4, moving the delivery member hub 14 from the proximal position to the distal position, thereby mounting a delivery member attached to the delivery member hub 14 to a medicament container with the housing 4 of the medicament delivery device.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are described in the clauses below.
1. A sub-assembly (1) of a medicament delivery device, the sub-assembly (1) comprising:
   a tubular outer cap (10) extending along a longitudinal axis (L);
   an inner cap (12) at least partially surrounded by the tubular outer cap (10); wherein the inner cap (12) is configured to be removably attached to a housing of the medicament delivery device via a rotational attachment;
   a clutch formed by a driving part (101) and a driven part (132);
   wherein the driven part (132) is attached to the inner cap (12) and positioned between the tubular outer cap (10) and the inner cap (12) in a direction transverse to the longitudinal axis (L);
   wherein the outer cap (10) is axially movable relative to the inner cap (12) between a proximal position and a distal position;
   wherein the tubular outer cap (10) comprises the driving part (101), the driving part (101) extending from a body (100) of the tubular outer cap (10); wherein the driven part (132), the driven part (132) extending in the direction transverse to the longitudinal axis towards the tubular outer cap (10); wherein the driving part (101) is spaced apart from the driven part (132) in the direction of the longitudinal axis (L) when the tubular outer cap (10) is in the proximal position; and wherein the driving part (101) is adjacent to the driven part (132) when the tubular outer cap (10) is in the distal position so that the inner cap can be detached from the housing of the medicament delivery device via a rotation of the outer cap (10) relative to the housing of the medicament delivery device.
2. The sub-assembly (1) according to clause 1, wherein the driven part extends from a body wall of the inner cap in the direction transverse to the longitudinal axis (L).
3. The sub-assembly (1) according to clause 1, the sub-assembly comprises an interlock sleeve (13); wherein the interlock sleeve (13) comprises a body (130) extending along the longitudinal axis (L); wherein the interlock sleeve (13) comprises a rotational sleeve fastener (133) engaged with a counter rotational sleeve fastener (121) of the inner cap (12); and wherein the driven part (132) extends from the body (130) of the interlock sleeve (13) in the direction transverse to the longitudinal axis (L).
4. The sub-assembly according to any one of the preceding clauses, wherein one of the driving part (101) and the driven part (132) is a recess; and wherein the other one of the driving part (101) and the driven part (132) is a protrusion positioned in the recess when the tubular outer cap (10) is in the distal position.
5. The sub-assembly according to clause 4, wherein the recess faces towards the direction transverse to the longitudinal axis.
6. The sub-assembly according to clause 4 or 5, wherein the recess is formed by a ratchet.
7. The sub-assembly according to any of the preceding clauses, wherein the clutch engagement formed between the driving part and the driven part is formed by a non-circular cross-section body of the driving part matching with a non-circular cross-section body of the driven part.
8. The sub-assembly according to any of the preceding clauses, wherein the clutch engagement is formed between the driving part and the driven part is a friction-fit engagement.
9. The sub-assembly according to any of the preceding clauses, the sub-assembly comprises and a retainer (11); wherein the retainer comprises a retainer body (110), and an attachment (11a) extending from the retainer body (110); wherein the attachment (11a) is configured to attach to a counter attachment of the medicament delivery device; wherein the inner cap (12) is removable attached to the retainer (11) via the rotational attachment, thereby the inner cap (12) is removable attached to the medicament delivery device via the retainer (11).
10. The sub-assembly according to clause 9, wherein the rotational attachment is formed by a screw thread (111) of the retainer body (110) and a thread follower (123) of the inner cap (12) that is engaged with the screw thread (111) of the retainer (11).
11. The sub-assembly according to clauses 9 or 10, the sub-assembly comprises a delivery member hub (14) configured to attach with a medicament delivery member; wherein the delivery member hub (14) comprises a first engaging member (141) engaged with a first counter engaging member (112) of the retainer (11); and a second engaging member (142) engaged with a counter second engaging member (124) of the inner cap (12); and wherein the delivery member hub (14) is axially movable relative to the retainer body (110) between a proximal position and a distal position.
12. The sub-assembly according to clause 11, wherein a rotation of the inner cap (12) relative to the retainer (11) is configured to move the delivery member hub (14) from the proximal position to the distal position.
13. The sub-assembly according to clause 12 dependent on clause 10, wherein an engagement formed between the first engaging member and the counter first engaging member is a screw thread engagement; wherein an engagement formed between the second engaging member and the counter second engaging member is rotational engagement; and wherein the screw thread engagement between the delivery member hub and the retainer is directed to a reverse longitudinal direction relative to the screw thread engagement between the inner cap and the retainer.
14. The sub-assembly according to clause 12, wherein the delivery member hub is rotationally fixed to the retainer body; wherein one of the second engaging member and the counter second engaging member is a helical cam and the other one of the second engaging member and the counter second engaging member is a cam follower; and wherein an engagement formed between the first engaging member and the counter first engaging member is a snap-fit engagement.
15. The sub-assembly according to any of clauses 9-14, wherein the attachment (11a) of the retainer (11) is one of the snap-fit attachment, a screw thread attachment and a bayonet attachment.
16. The sub-assembly according to any of the preceding clauses, the sub-assembly comprises a delivery member guard (2) and a lock member (3); wherein the delivery member guard (2) comprises a proximally directed surface (20a) adjacent to a distally directed surface (102) of the tubular outer cap (10); wherein the delivery member guard (2) comprises a distally directed surface (22a); wherein the lock member (3) comprises a body (30) having a proximally directed surface (31a); wherein the lock member (3) is movable relative to the delivery member guard (2) between a locked position where the proximally directed surface (31a) of the lock member (3) is aligned with the distally directed surface (22a) of the delivery member guard (2) so that the delivery member guard (2) is prevented from moving distally relative to the lock member (3) and an unlocked position where the proximally directed surface (31a) of the lock member (3) is offset to the distally directed surface (22a) of the delivery member guard (2) so that the delivery member guard (2) is distally movable relative to the lock member (3).
17. The sub-assembly according to clause 16, wherein the lock member (3) is rotatable relative to the delivery member guard (2) about the longitudinal axis (L) between the locked position and the unlocked position.
18. The sub-assembly according to clause 16, wherein the lock member is slidable relative to the delivery member guard in the direction transverse to the longitudinal axis.
19. A medicament delivery device comprising the sub-assembly according to any of the preceding clauses, wherein the medicament delivery device is an autoinjector.
20. A cassette unit (C) of a medicament delivery device, the cassette unit comprising the sub-assembly according to clause 17 or 18, wherein the cassette unit comprises the housing (4) of the medicament delivery device; wherein the housing (4) extending along the longitudinal axis (L); wherein the lock member (3) is axially fixed to the housing (4).
21. The cassette unit (C) according to clause 20 when dependent on clause 17, wherein the delivery member guard (2) is rotationally fixed to the housing (4), wherein the housing (4) comprises a bayonet protrusion (40) configured to move along a bayonet track of a drive unit (R) of the medicament delivery device thereby attaching the cassette unit (C) to the drive unit (R); wherein the lock member (3) comprises a protrusion (32) extending from the body (30) away from the longitudinal axis (L); wherein the protrusion (32) is next to the bayonet protrusion (40) of the housing (4) in the direction of the longitudinal axis (L); and wherein the protrusion (32) is configured to be rotationally fixed to a wall of the drive unit (R) when the cassette unit is attached to the drive unit (R).
22. A method of cap removal of a medicament delivery device, wherein the medicament delivery device comprises the cassette unit according to clause 20 and a drive unit, the method comprising the following steps in the following order:
   moving the lock member from the locked position to the unlocked position;
   moving the tubular outer cap with the delivery member guard in the distal direction relative to the inner cap to the distal position of the tubular outer cap;
   rotating the tubular outer cap with the inner cap relative to the housing of the cassette unit, thereby detaching the tubular outer cap and the inner cap from the housing of the cassette unit.
23. The method according to clause 22 when the medicament delivery device comprises the cassette unit according to clause 21, wherein the step of moving the lock member from the locked position to the unlocked position comprising the step of:
   rotating the lock member relative to the delivery member guard from the locked position to the unlocked position.
24. The method according to clause 23, wherein the step of rotating the lock member relative to the delivery member guard from the locked position to the unlocked position comprises the step of:
   rotating the lock member relative to the delivery member guard from the locked position to the unlocked position during attaching the cassette unit to a drive unit via a bayonet connection.
25. The method according to any of clauses 22-23 when the cassette unit comprises the sub-assembly according to any of clauses 11-14, wherein the step of rotating the tubular outer cap with the inner cap relative to the housing comprises the further step of:
   during rotating the tubular outer cap with the inner cap relative to the housing, moving the delivery member hub from the proximal position to the distal position, thereby mounting a delivery member attached to the delivery member hub to a medicament container with the housing of the medicament delivery device.

1-15. (canceled)

16. A sub-assembly of a medicament delivery device, the sub-assembly comprising:
a tubular outer cap extending along a longitudinal axis;
an inner cap at least partially surrounded by the tubular outer cap, wherein the inner cap is configured to be removably attached to a housing of the medicament delivery device via a rotational attachment; and
a clutch formed by a driving part and a driven part,
wherein the driven part is attached to the inner cap and positioned between the tubular outer cap and the inner cap in a direction transverse to the longitudinal axis,
wherein the outer cap is axially movable relative to the inner cap between a proximal position and a distal position,
wherein the tubular outer cap comprises the driving part, the driving part extending from a body of the tubular outer cap,
wherein the driven part, the driven part extending in the direction transverse to the longitudinal axis towards the tubular outer cap,
wherein the driving part is spaced apart from the driven part in the direction of the longitudinal axis when the tubular outer cap is in the proximal position, and
wherein the driving part is adjacent to the driven part when the tubular outer cap is in the distal position so that the inner cap can be detached from the housing of the medicament delivery device via a rotation of the outer cap relative to the housing of the medicament delivery device.

17. The sub-assembly according to claim 16, the sub-assembly comprises an interlock sleeve, wherein the interlock sleeve comprises a body extending along the longitudinal axis, wherein the interlock sleeve comprises a rotational sleeve fastener engaged with a counter rotational sleeve fastener of the inner cap, and wherein the driven part extends from the body of the interlock sleeve in the direction transverse to the longitudinal axis.

18. The sub-assembly according to claim 16, wherein one of the driving part and the driven part is a recess, and wherein the other one of the driving part and the driven part is a protrusion positioned in the recess when the tubular outer cap is in the distal position.

19. The sub-assembly according to claim 18, wherein the recess faces towards the direction transverse to the longitudinal axis.

20. The sub-assembly according to claim 16, the sub-assembly comprises and a retainer, wherein the retainer comprises a retainer body and an attachment extending from the retainer body, wherein the attachment is configured to attach to a counter attachment of the medicament delivery device, wherein the inner cap is removable attached to the retainer via the rotational attachment, thereby the inner cap is removable attached to the medicament delivery device via the retainer.

21. The sub-assembly according to claim 20, wherein the rotational attachment is formed by a screw thread of the retainer body and a thread follower of the inner cap that is engaged with the screw thread of the retainer.

22. The sub-assembly according to claim 20, the sub-assembly comprises a delivery member hub configured to attach with a medicament delivery member, wherein the delivery member hub comprises a first engaging member engaged with a first counter engaging member of the retainer, and a second engaging member engaged with a counter second engaging member of the inner cap, and wherein the delivery member hub is axially movable relative to the retainer body between a proximal position and a distal position.

23. The sub-assembly according to claim 22, wherein a rotation of the inner cap relative to the retainer is configured to move the delivery member hub from the proximal position to the distal position.

24. The sub-assembly according to claim 23, wherein an engagement formed between the first engaging member and the counter first engaging member is a screw thread engagement, wherein an engagement formed between the second engaging member and the counter second engaging member is rotational engagement, and wherein the screw thread engagement between the delivery member hub and the retainer is directed to a reverse longitudinal direction relative to the screw thread engagement between the inner cap and the retainer.

25. The sub-assembly according to claim 23, wherein the delivery member hub is rotationally fixed to the retainer body, wherein one of the second engaging member and the counter second engaging member is a helical cam and the other one of the second engaging member and the counter second engaging member is a cam follower, and wherein an engagement formed between the first engaging member and the counter first engaging member is a snap-fit engagement.

26. The sub-assembly according to claim 20, wherein the attachment of the retainer is one of the snap-fit attachment, a screw thread attachment and a bayonet attachment.

27. The sub-assembly according to claim 16, the sub-assembly comprises a delivery member guard and a lock member, wherein the delivery member guard comprises a proximally directed surface adjacent to a distally directed surface of the tubular outer cap, wherein the delivery member guard comprises a distally directed surface, wherein the lock member comprises a body having a proximally directed surface, wherein the lock member is movable relative to the delivery member guard between a locked position where the proximally directed surface of the lock member is aligned with the distally directed surface of the delivery member guard so that the delivery member guard is prevented from moving distally relative to the lock member and an unlocked position where the proximally directed surface of the lock member is offset to the distally directed surface of the delivery member guard so that the delivery member guard is distally movable relative to the lock member.

28. The sub-assembly according to claim 27, wherein the lock member is rotatable relative to the delivery member guard about the longitudinal axis between the locked position and the unlocked position.

29. A cassette unit of a medicament delivery device, the cassette unit comprising the sub-assembly according to claim 28, wherein the cassette unit comprises the housing of the medicament delivery device, wherein the housing extending along the longitudinal axis, and wherein the lock member is axially fixed to the housing.

30. The cassette unit according to claim 29, wherein the delivery member guard is rotationally fixed to the housing, wherein the housing comprises a bayonet protrusion configured to move along a bayonet track of a drive unit of the medicament delivery device thereby attaching the cassette unit to the drive unit, wherein the lock member comprises a protrusion extending from the body away from the longitudinal axis, wherein the protrusion is next to the bayonet protrusion of the housing in the direction of the longitudinal axis, and wherein the protrusion is configured to be rotationally fixed to a wall of the drive unit when the cassette unit is attached to the drive unit.
